(19) United States
(12) Patent Application Publication
(10) Pub. No.: US 2017/0188799 A1
(43) Pub. Date: Jul. 6, 2017

(54) CATHETER FOR TRACHEO-BRONCHIAL SUCTION WITH VISUALIZATION MEANS

(71) Applicants: Mario Zocca, Verona (IT); TECRES S.p.A., Sommacampagna (Verona) (IT)

(72) Inventors: Mario Zocca, Verona (IT); Giovanni Faccioli, Monzambano (IT); Renzo Soffiatti, Nogara (IT)

(21) Appl. No.: 15/463,083

(22) Filed: Mar. 20, 2017

Related U.S. Application Data
(63) Continuation-in-part of application No. 13/148,561, filed on Aug. 18, 2011, now Pat. No. 9,603,515.

(51) Int. Cl. A61B 1/015; A61M 25/00; A61B 1/005; A61B 1/00; A61B 1/267; A61M 1/00; A61B 1/07

(52) U.S. Cl. CPC A61B 1/015; A61M 1/008; A61M 25/005; A61B 1/07; A61B 1/00163; A61B 1/2676; A61B 1/00066; A61B 1/00128; A61B 1/0051

(57) ABSTRACT

A catheter for tracheal or bronchial suction including a unit for displaying the artificial airways, the trachea and the bronchial tree so as to allow the removal of secretions; in particular, the catheter for tracheal or bronchial suction includes optical fibers, a microcamera or another visualization technology positioned in the distal end. The operator can identify the position of the distal end, of the artificial airways, of the trachea and of the bronchial tree on the screen. Therefore it is possible to ensure that the tube of the catheter is adjacent to or inside collections of fluid secretions or other material to be sucked up. The suction can be selectively applied to avoid damage to the mucosa and to facilitate the more complete elimination of secretions.

### CROSS-REFERENCE TO RELATED APPLICATIONS

This is a continuation-in-part of co-pending application Ser. No. 13/148,561 filed Aug. 18, 2011 which is the U.S. National Stage of International Application No. PCT/IB2010/050590 filed on Feb. 9, 2010, the disclosure of which is incorporated herein by reference.

### TECHNICAL FIELD OF THE INVENTION

The present invention concerns a catheter for tracheo-bronchial suction, especially of the disposable type, with visualization means, i.e. a catheter for tracheal or bronchial suction equipped, in the distal end to be introduced into the patient, with means for displaying the artificial airways and/or the trachea and/or the bronchial tree.

### STATE OF THE ART

Patients kept alive through mechanical ventilators or artificial respirators often require an artificial airway, for example a translaryngeal, tracheostomy or supraglottic tube.

Generally, these patients accumulate fluid secretions in the trachea or in the bronchial tree.

These secretions require periodic removal, which is normally carried out through a procedure that involves: passing a catheter with a small diameter down through the artificial airway in the trachea or in the bronchial tree, applying suction to the catheter through which the secretions are transferred along the inner lumen of the catheter to a collection container.

This procedure is carried out blind without any possibility of displaying and lighting up the position of the distal tip of the catheter.

The state of the art is described in American patents U.S. Pat. No.-4,569,344 and U.S. Pat. No.-4,351,328.

U.S. Pat. No.-4,569,344 describes a device and method for ventilation and suction that integrates in a single unit the selective ability of respiration and of sucking up fluids from the trachea and from the bronchi of a patient.

U.S. Pat. No.-4,351,328 describes another device and method for endotracheal suction of a patient without having to remove the patient from the respirator. A suction tube is positioned in the tracheal tube through a wall of the group of tubes of the respirator, so as to keep the respiration system intact.

However, the devices and methods described in these documents are problematic because they do not allow the patient's secretions to be completely removed.

In particular, it is difficult to selectively position the catheter in the desired region of the tracheo-bronchial tree and suction directly on the tracheo-bronchial mucosa can cause damage to the patient.

### SUMMARY OF THE INVENTION

The technical task of the present invention is therefore to make a catheter for tracheo-bronchial suction that can be easily introduced in selected areas of the trachea or of the bronchial tree.

In such a technical task, a purpose of the present invention is to make a catheter for tracheo-bronchial suction equipped with a visualization unit that is able to be separated from the catheter.

Such a task and such purposes are all accomplished with a catheter for tracheo-bronchial suction according to the present specification.

### BRIEF DESCRIPTION OF THE DRAWINGS

These and further advantages will be better understood by any man skilled in the art from the following description and from the attached drawings, given as a non-limiting example, in which:

FIG. 1 shows a rear view of a catheter for tracheo-bronchial suction according to the present invention;

FIG. 2 shows a side view of the catheter for tracheo-bronchial suction according to FIG. 1;

FIG. 3 shows a front view of the catheter for tracheo-bronchial suction according to the previous figures;

FIG. 4 shows a front view of the catheter for tracheo-bronchial suction according to the previous figures without the visualization unit;

FIG. 5 shows a side view of the catheter for tracheo-bronchial suction according to FIG. 4;

FIG. 6 shows a rear view of the catheter for tracheo-bronchial suction according to FIGS. 4 and 5;

FIG. 7 shows a schematic side section view of the catheter for tracheo-bronchial suction according to the previous figures;

FIGS. 8 and 9 show some details of the connection between the catheter and the visualization unit;

FIGS. 10 and 11 shows two sections of the catheter according to the present invention taken close to the distal tip;

FIGS. 12 and 13 show two sections of the catheter according to the present invention taken in the central portion of the catheter;

FIG. 14 shows another section of a tubular catheter according to the present invention removed from a respective viewer;

FIG. 15 shows a front view of another embodiment of catheter unit for tracheo-bronchial suction according to the present invention;

FIG. 16 shows the catheter unit of FIG. 15 in an exploded view;

FIG. 17 shows a detail in an enlarged view of the catheter unit of FIG. 15;

FIG. 18 shows another detail in an enlarged view of the catheter unit of FIG. 15;

FIG. 19 is an exploded view of the components illustrated in FIG. 17; and

FIG. 20 shows a detail in an enlarged view of the catheter unit of FIG. 15.

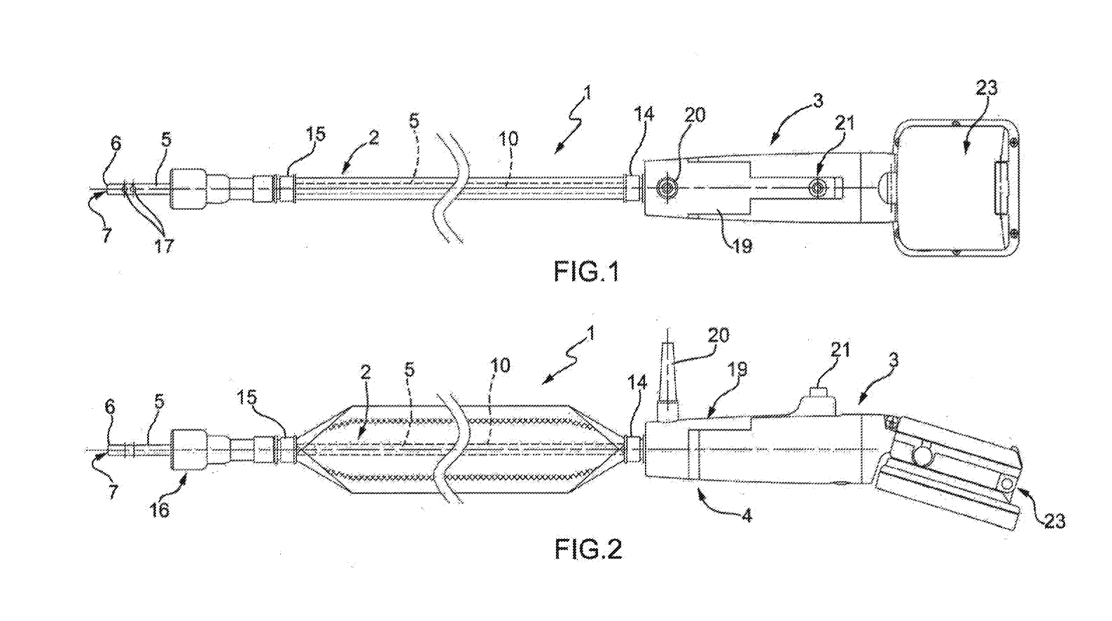

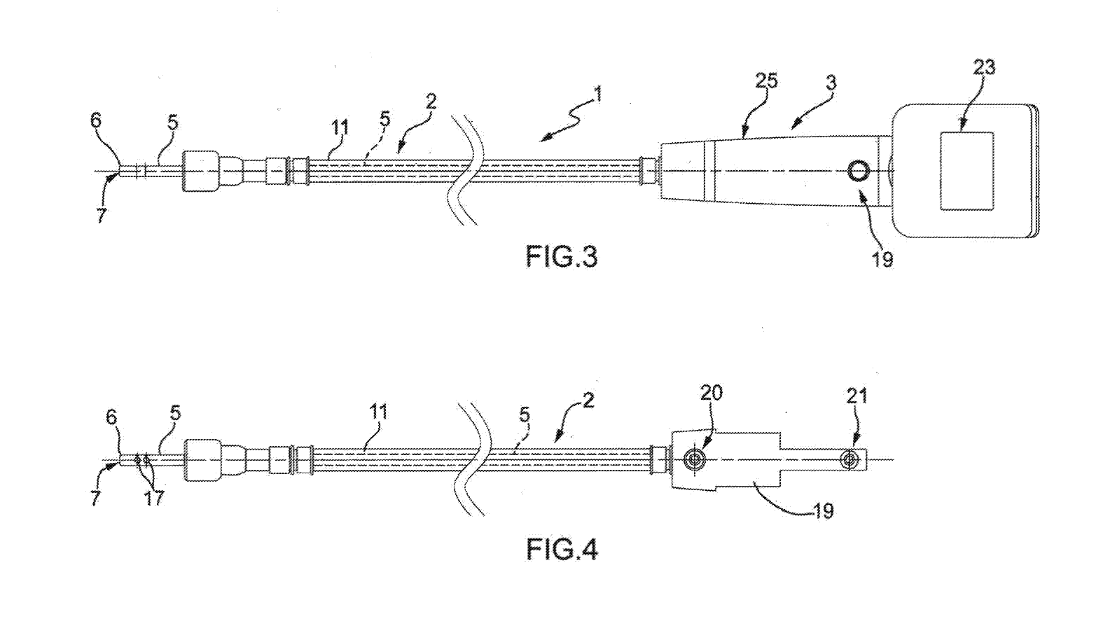

### DETAILED DESCRIPTION

With reference to the drawings, reference numeral 1 wholly indicates the catheter unit for tracheo-bronchial suction according to the present invention comprising a tubular group 2, in particular of the disposable type, and optionally a visualization unit 3 connected to the tubular group 2 through connection means 4.

The tubular group 2 comprises a tubular catheter 5 that comprises a distal end 6 adapted to be introduced into the trachea and/or into the bronchi of a patient to suck up fluid secretions or other similar material needing to be sucked up.

The distal end 6 is therefore rounded and equipped with an opening 7 connected through a tubular channel 10 to suction means suitable for sucking up fluid secretions and/or other similar material that can be found in a patient. In order to increase the possibility of removal of secretions the distal end 6 can also comprise one or more side holes 17 connected to the tubular channel 10.

The catheter unit 1 preferably comprises viewing means 8 and lighting means 9 extending through a channel or a respective channel delimited by the tubular catheter 5, and more particularly extending up to distal end 6 of the tubular catheter 5.

The viewing means 8 can comprise one or more optical fibers, cables or the like suitable for transferring an image, and the lighting means 9 can comprise other optical fibers suitable for guiding light; the aforementioned optical fibers are arranged in one or more tubular channels parallel to the channel 10 through which the fluid secretions pass. Preferably, all such channels are delimited by walls or parts of walls of the tubular catheter 5.

According to another version of the present invention the viewing means 8 can comprise a micro-camera arranged in the distal end 6, and the lighting means 9 can comprise one or more high-efficiency LEDs also arranged in the distal end 6. In this version, there are some cables for supplying power and transmitting images arranged in one or more tubular channels parallel to the channel 10 through which the fluid secretions pass.

Both for the optical fibers and for the micro-camera there is provided a lens made, for example, from plastic.

Moreover, other equivalent technologies can be used for capturing and transferring images and for lighting.

As it is clear from the figures, it can be seen that the section of the channel 10 through which the fluid secretions pass is much larger than the section of the channels for the transmission of images and for lighting and it is practically slightly smaller than the external section of the catheter.

In particular, the tubular catheter 5 that is introduced into the patient has minimal dimensions, for example it has a diameter of between 4 mm and 8 mm, and therefore it can be withstood by almost all patients. Moreover, the tubular catheter 5 can have a length ranging between 500 mm and 1000 mm, for example about 800 mm.

Moreover, in the disposable version it is always ready for use on patients, thus avoiding laborious and expensive sterilisation processes.

The tubular group 2 can comprise a fixed or removable sheath or outer coating 11 to keep the tubular catheter 5 not contaminated by external agents during the insertion into or removal from the patient.

In other words, it is possible to carry out many insertions- and removals- of the tubular catheter 5, for example removing the fluid secretions from a patient on many successive occasions, keeping the tubular catheter 5 protected by the coating 11 and thus keeping it not contaminated by external agents even during removal.

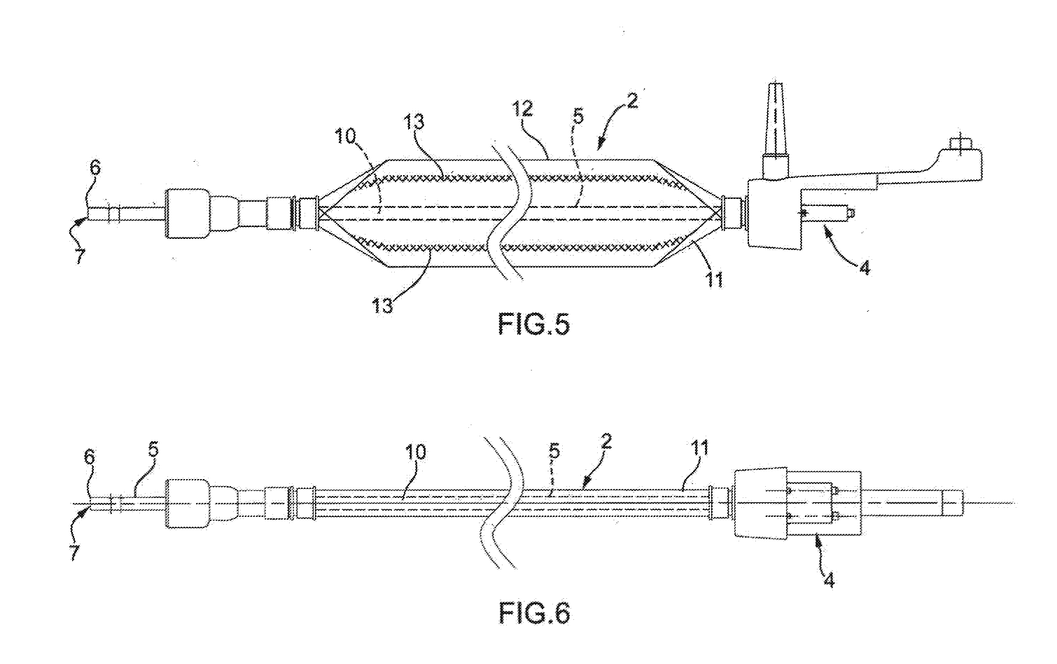

According to what is illustrated in FIG. 5, the coating 11 comprises a film 12 that is bent in a tubular shape and welded along a longitudinal welding zone 13.

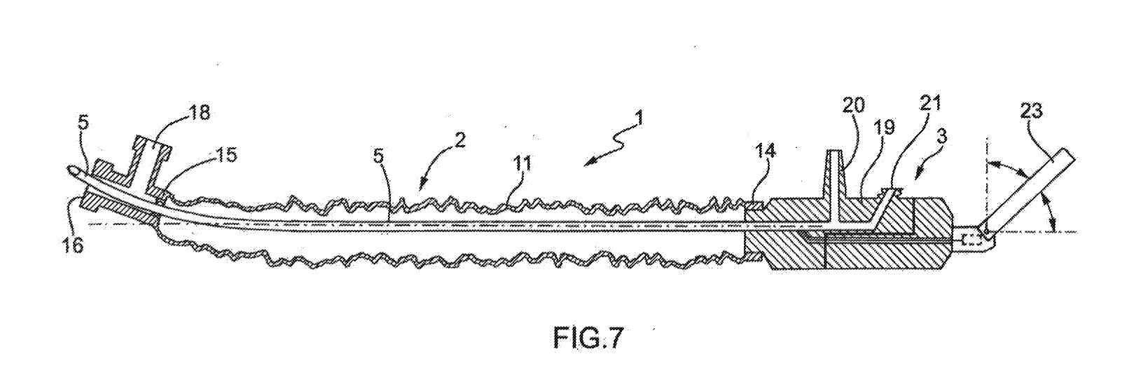

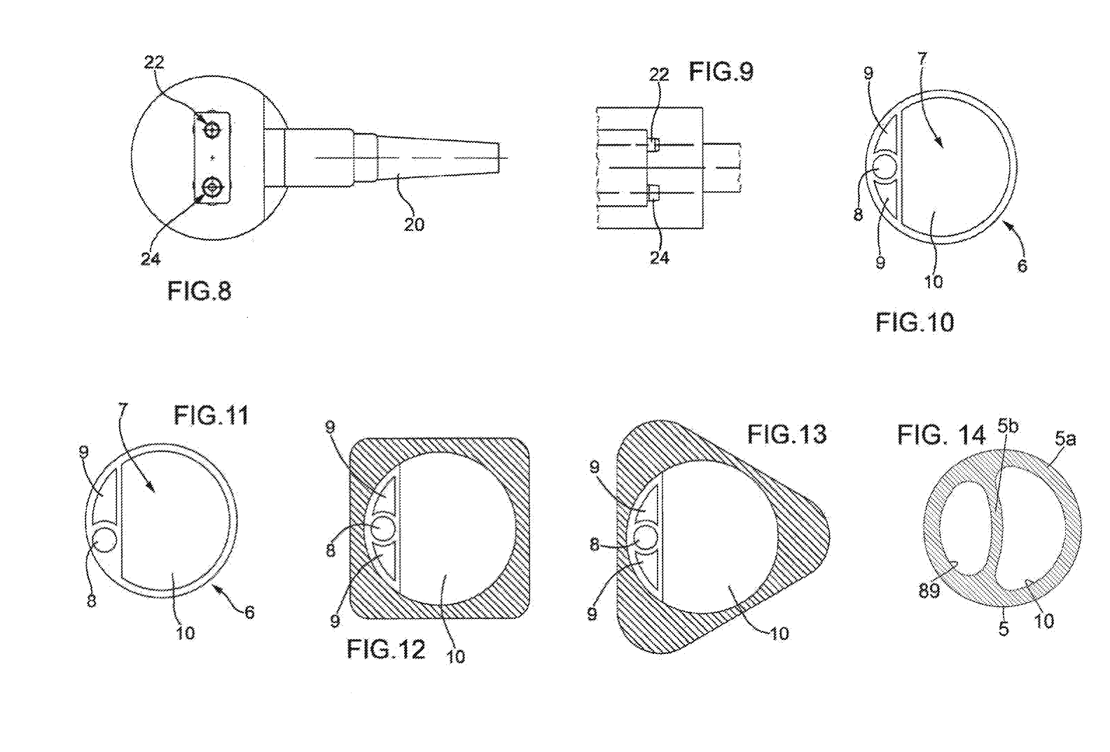

According to what has been illustrated more specifically in FIG. 7, the coating 11 is connected to the ends 14, 15 so as to completely enclose the tubular catheter 5 in the connection area 3 to the visualization unit and to the duct with the connection 16 to the tracheal tube and the connection 18 to the ventilation circuit. It must be kept in mind that the casing 11 and the duct with the connection 18 to the ventilation circuit even if illustrated in the figures are completely optional.

Therefore, the catheter unit 1 for tracheo-bronchial suction according to the present invention, in its most simple version and with regard to the part to be introduced into the patient, can comprise just the tubular catheter 5.

The tubular group 2 comprises a portion 19 equipped with an attachment 20 for suction means, for example a vacuum source, and a hole 21 able to be closed up by the operator to activate the suction. The portion 19 also comprises the connection means 4 that allow a safe connection of the tubular group 2 to the visualization unit 3 to be obtained.

In the disposable version, the entire tubular group 2 comprising the tubular catheter 5, the portion 19 and the connection means 4, is removed and replaced each time the sterility conditions require it, and it should be emphasised that such a replacement also involves the viewing means 8 and the lighting means 9, i.e., according to the versions, the optical fiber or fibers or cable/s suitable for transferring an image and the optical fibers suitable for guiding light or else the micro-camera and the high-efficiency LEDs or possible other viewing and lighting devices, including the relative lens.

In any case, such elements are relatively cost-effective and can be replaced each time together with the tubular catheter 5.

The visualization unit 3 is of the type that can be reused many times and comprises a viewer 23 that can be tilted or pivoted so that the operator can see it better. In the case in which the transmission of images and light takes place through optical fibers, the visualization unit 3 also comprises a micro-camera with a CMOS colour sensor and at least a high-efficiency white LED arranged close to the connection means 4.

The connection means 4 can comprise two attachment elements 22, 24, and such attachment elements 22, 24, being comprised in the connection means 4, are replaced together with the tubular group 2, when in the disposable version this group is replaced.

An important characteristic of the connection means 4 consists of the shape of the two attachment elements 22, 24.

An attachment element 22 has a first substantially cylindrical portion and a conical or tapered end portion. The attachment element 22 inserts into a matching seat foreseen in the visualization unit 3, and the shape of this element is such as to allow optimal centring of the image coming from the optical fiber with respect to the microcamera present in the visualization unit 3.

The other attachment element 24 has a frusto-conical attachment that inserts into a corresponding frusto-conical seat foreseen in the visualization unit 3, and in this case the shape is optimal to avoid light dispersions.

According to a version of the present invention, the visualization unit 3 comprises a connection for a module with a power supply unit and a wireless or Bluetooth functionality to allow the images to be seen on a separate or remote screen. The visualization unit 3 can also be used with a plurality of other medical devices.

The set of the portion 19 and the part of the visualization unit 3 that connects to it constitute a handle 25 by means of which the operator can comfortably grip and orient the catheter.

An important characteristic of the present invention is indeed given by the possibility of orienting and directing the tubular catheter 5 selectively in the trachea and/or the bronchi.

According to a version of the present invention, the tubular catheter 5 has a curvature along the longitudinal axis of the catheter, for example in an intermediate position or close to the distal end 6, to allow the end of the catheter to be directed by rotating the catheter around its longitudinal axis.

According to another version of the present invention, the tubular catheter 5 has a mechanism for guiding the distal end 6 of the catheter.

According to yet another version of the present invention, the tubular catheter 5 has a guiding mechanism through which the catheter is made with a certain curvature and there are means for straightening the catheter that use a chuck, a wire, a tube or a rectilinear shaped reinforcer that can be reversibly inserted along the tubular catheter 5 or in a separate channel inside the tubular catheter itself.

According to a further version of the present invention, the tubular catheter 5 is entirely or partially made with a material that has a permanent or temporary memory, so that the tube can be modelled, before insertion into the patient.

In order to effectively transmit the rotation to the distal end 6, the tube of the tubular catheter 5 has special constructive characteristics.

The distal end 6 is relatively flexible and has a tubular section with relatively thin walls. The central and proximal part of the tube of the tubular catheter 5 is more rigid against twisting so as to transmit the rotations applied to the user through the handle 25.

In order to obtain the greater rigidity of the central and proximal part, the tube of the tubular catheter 5 can be made with a greater thickness in such central and proximal parts, for example by wrapping it on the outside with another tube for a certain longitudinal portion, or else by adding material during the manufacturing process, for example during extrusion, so as to obtain a slightly frusto-conical shape, or else by loading the material of the tube with materials capable of stiffening the structure especially with respect to twisting. Moreover, the tube can be made with greater thickness and therefore mechanically machined to obtain a slightly frusto-conical shape.

As an alternative or in combination with the above-mentioned solutions, the tubular catheter 5 could be provided with reinforcement meshes or grids, for example hidden or not visible or revealable outside the tubular catheter 5 itself.

Such mesh or meshes could be fitted in the tubular catheter 5 or inside the same, i.e. embedded therein or in the matrix thereof. The mesh or meshes can be for example shaped as a tubular member with axis substantially corresponding to the main longitudinal axis of the tubular catheter 5 or can be shaped as the outer and inner walls of the tubular catheter 5.

Preferably, the mesh or meshes does/do not protrude outside, so as to prevent any patient tissues or parts to be damaged, and/or inside the tubular catheter 5, so as to prevent any alteration of the optical signals transmitted by the viewing or lighting means.

The mesh or meshes is/are designed to improve the anti-torsion or anti-twisting property of the tubular catheter 5, although the mesh or meshes does/do not prevent or limit the flexion or bending properties of the tubular catheter 5.

Moreover, the mesh or meshes can be of medical grade and with constant mesh size.

The mesh or meshes can be extruded together with the matrix forming the tubular catheter, which matrix can be for example made of silicone, PVC, PU and TPE.

The mesh or meshes can instead be made of metal, polymer or of another suitable material.

According to what has been illustrated in FIGS. 12 and 13 the shape in section can also be varied, for example with a square or triangular shape with rounded corners.

In this case, the tube is made firstly in a cylinder shape and then is heat moulded in moulds with a square or triangular section with rounded corners.

So far as FIG. 14 is concerned, the same shows a cross-section view of another embodiment of tubular catheter 5 for a catheter unit 1 according to the present invention. Such tubular catheter 5, more particularly for the whole extension or length thereof, delimits two channels 10 and 89 only, preferably substantially parallel to one another, i.e. a first channel 10 designed to be connected to suction means suitable for sucking up fluid secretions and/or other similar material that can be found in a patient, and a second channel 89 housing or suitable for housing viewing means 8 and/or lighting means 9.

The first channel 10 has a section substantially equal or greater than the second channel 89, for example the first channel 10 has a cross-section about 10-90%, for example 10-20% or 30-50% or 70-90% greater than the second channel 89.

More particularly, the tubular catheter 5 includes an outer wall 5a with a cross-section shaped as a circle or polygon and further includes an inner wall 5b bridge extending from a first part of the inner face of the outer wall 5a to another part thereof. The inner wall 5b can be shaped in cross-section as a curve member or with a number of sections each inclined with respect to the adjacent/s.

Thus, each surface of the inner wall 5b delimits together with a corresponding portion of the inner face of the outer wall 5a the first 10 or the second channel 89.

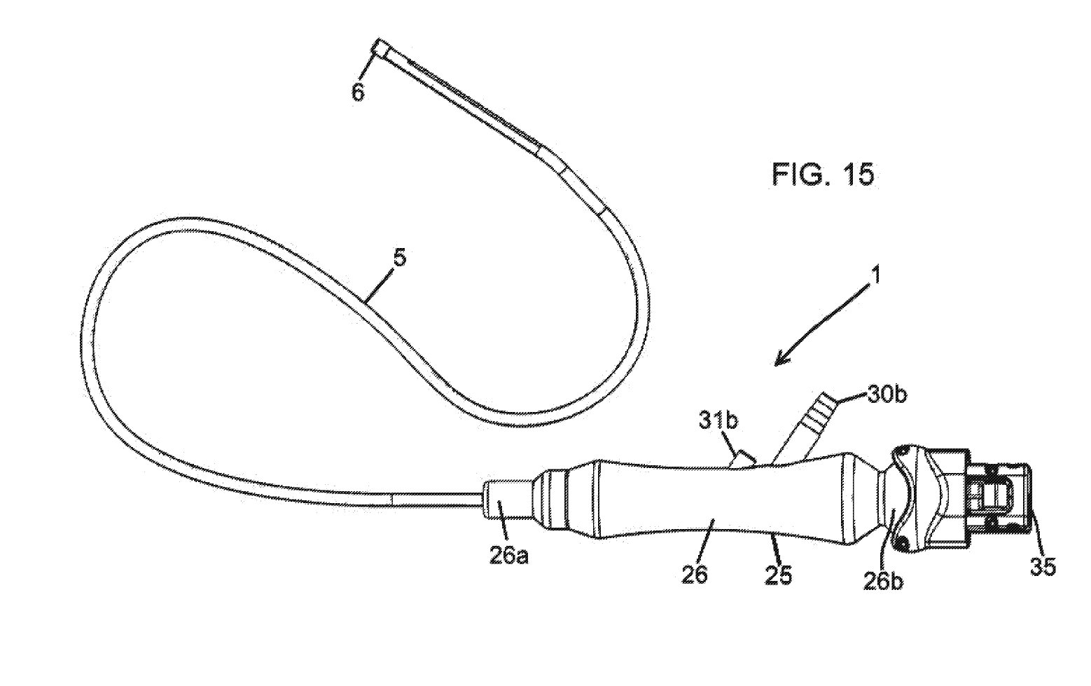

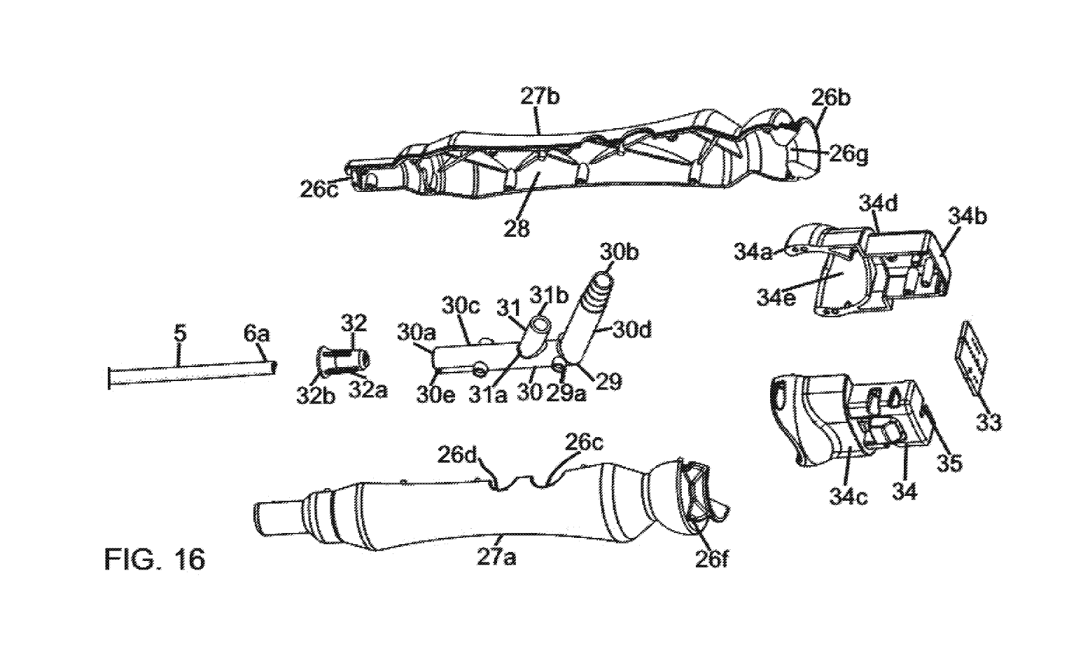

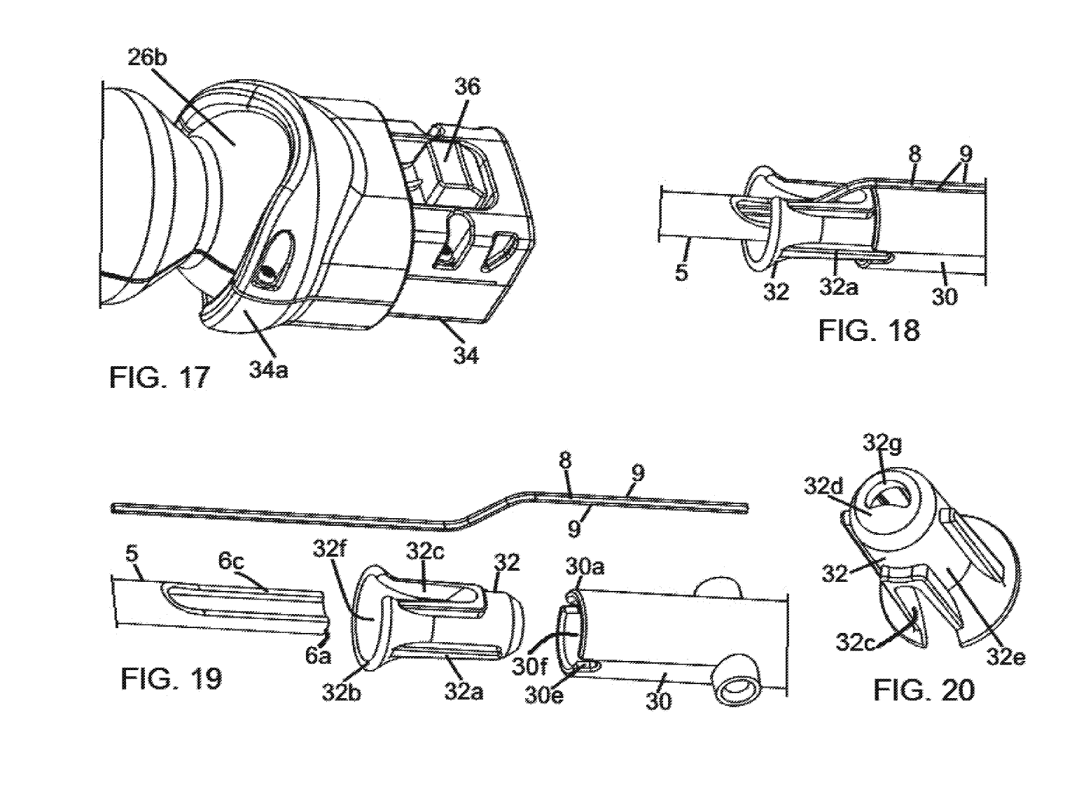

Referring now to FIGS. 15 to 20, a catheter unit 1 for tracheo-bronchial suction according to another embodiment of the present invention is shown, that can include one or more of the features of the present invention described above, which catheter unit 1 includes a handle 25 comprising a housing component 26, for example consisting of two shells or hemi-shell members 27a, 27b connectable to one another, for example through connecting means, e.g. snap-fitting means and/or screws or the like and delimiting a main seat 28 for receiving a connector component 29.

As soon as the two shells or hemi-shell members 27a, 27b are engaged with one another, the same or respective projections, for example projections extending inside the main seat 28 or at ends of the hemi-shell members 27a, 27b can constrain or fix in position one or more components of the catheter unit 1, such as the tubular catheter 5.

Moreover, the housing component 26 delimits at a first end 26a thereof an insertion opening 26c for the insertion of the tubular catheter 5, whereas its other or second end 26b is suitable for being connected to a viewer or to a joint or to a junction component for the connection to a viewer 23.

The connector component 29 can include a tubular member comprising a main tubular body 30 having a first end 30a of insertion and constrain of the tubular catheter 5 and a second end 30b to be connected in fluid communication with suction means, for example a vacuum source, as well as a branch tubular body 31 extending from the main tubular body 30 and including a first extremity 31a opening at the through hole delimited by the main tubular body 30 and a second extremity 31b, which, in use, opens outside the housing component 26 and can be closed up, for example by an operator as soon as the suction means are activated or slightly before or after such a step. The tubular member including the main tubular body 30 and the branch tubular body 31 is preferably made enbloc or in a single piece.

The main tubular body 30 can be L-shaped or with two sections inclined with respect to each other, a first section 30c opening at the first end 30a extending about an axis substantially coinciding or parallel to the main longitudinal axis of the housing component 26 and a second section 30d inclined with respect to the first section 30c for an angle or about 15-60°, or preferably 30-45° and opening at the second end 30b.

The tubular member 30, 31 or the housing component 26 could also be provided with spacers or positioning elements 29a designed to keep the tubular member 30, 31 in a fixed or stable position inside the seat 28 as soon as the same is fitted into the housing component 26 or the latter is closed or the two shells or hemi-shell members 27a, 27b are assembled with one another.

The housing component 26 at portions of its periphery can delimit respective second 26d and third 26e seats for the passage of the part of the main tubular body 30 defining the second end 30b and for the part of the branch tubular body 31 delimiting the second extremity 31b.

The connector component 29 could also be provided with a bushing component 32 to be fitted, for example to size, or fixed into the first end 30a of the main tubular body 30 and designed to clamp a respective proximal end 6a of the tubular catheter 5, for example as soon as it (the bushing component 32) is inserted into such first end 30a.

In this respect, the bushing component 32 could be wholly or partly tapered with cross section decreasing in a direction from the first end 30a to the second end 30b, i.e. in the direction of insertion or fitting with the main tubular body 30.

Moreover, the main tubular body 30 and the bushing component 32 can be provided with mutual connection or fixing means, e.g. ribs 32a of the bushing component 32 or of the main tubular body 30 to be slidingly partly or wholly inserted into slots 30e delimited by the other among main tubular body 30 and bushing component 32. The ribs 32a can also be suitable for providing a guided insertion or connection of the bushing component 32 into/with the main tubular body 30, this being required in particular if the bushing component 32 includes a groove 32c as will be described in the following.

The bushing component 32 can also be provided with an open or through end 32b for the insertion of the proximal end 6a of the tubular catheter 5.

The bushing component 32 could have a C-shaped cross-section for whole or part of its extension. Referring to the embodiment shown in the figures, the bushing component 32 delimits a groove 32c extending from one end, more particularly from the end distal from the second end 30b of the main tubular body 30, to an intermediate part thereof.

More particularly, the bushing component 32 includes a proximal or base wall 32d to be firstly inserted or fitted into the first end 30a of the main tubular body 30 or distal from the distal end 6 of the tubular catheter 5 and a side tubular wall 32e protruding from the proximal or base wall 32e and integral or made enbloc therewith, the free end of the side tubular wall 32e defining the open or through end 32b.

The side tubular wall 32d further delimits a through longitudinal light 32f designed to house or enclose the proximal end 6a of the tubular catheter 5, whereas the proximal or base wall 32e delimits a transfer through hole 32g with a section corresponding to and entirely aligned, in use, with the first channel 10 only, so that the connector component 29 or better yet the through hole 30f delimited by the main tubular body 30 is in fluid communication with the first channel 10 only, or in any case only with the channel delimited by the tubular catheter 5 and designed to be connected to suction means and suitable for sucking up fluid secretions and/or other similar material that can be found in a patient.

So far as the viewing means 8 and the lighting means 9 are concerned, the same could be provided within the seat 28 of the housing component 26, but preferably extending outside the connector component 29.

In this respect, the viewing means 8 and the lighting means 9 extend from one or a respective control board 33 (for example housed into the seat 28 or in a junction component) into the tubular catheter 5 and up to the distal end 6 thereof. The control board/s 33 can be provided with a lighting source, such as one or more LEDs designed to generate light to be transferred by means of the optical fibers 9. Moreover, the control board/s 33 could include a battery or battery means.

Furthermore, the control board/s 33 can be provided with electronic components suitable for transferring via wireless or Bluetooth the images obtained through the viewing means 8 to a separate or remote screen.

Referring to the not limiting embodiment shown in the figures, the viewing means 8 include a cable or an optical sensor, whereas the lighting means 9 include one or more, preferably two, three of four optical fibers 9 surrounding or running around or flanked to the viewing means 8 for the whole extension thereof. In this respect, connecting means, such as glue, clamping band/s or the like for connecting or constraining the viewing means 8 to the lighting means 9 can also be provided.

The group or bundle including viewing means 8 and lighting means 9 can be entered into the proximal end 6a of the tubular catheter 5 passing throughout the seat 28, preferably without entering the connector component 29; for example such a bundle can be entered into the proximal end 6a through the groove 32c of the bushing component 32, before or after the latter has been assembled with the connector component 29. To this regard, the bushing component 32 accomplishes also the task of guiding the viewing means 8 and the lighting means 9 so as to prevent the same from being damaged owing to an excessive or wrong inclination. In this respect, the proximal end 6a could be partly sectioned at part of the outer wall 5a delimiting the second channel 89, in such a way as to provide a huge opening 6c for the insertion of the viewing means 8 and the lighting means 9 into the second channel 89.

Referring now to the connection of the handle 25 to the visualization means or viewer 23 or, if provided of the housing component 26 to the visualization means or viewer 23, the same can be obtained through a pivoting or ball or spherical joint. Such a ball joint makes it possible to rotate the viewer 23 with respect to the handle 25 or to the housing component 26 and, in turn, to the tubular catheter 5 substantially in all directions.

To this regards, the second end 26b of the housing component 26 could be at least partly spherically or hemi-spherically shaped or delimiting an at least partly spherically or hemi-spherically shaped pivoting seat for the connection to a respective end of the visualization means or viewer 23.

As an alternative, a junction component 34 can be provided between the housing component and the visualization means, which junction component 34 includes a first end 34a to be coupled with the second end 26b of the housing component 26, for example in such a way as to obtain a ball joint connection therebetween.

The second end 26b of the housing component 26 or, if provided, the junction component 34 could include means of removable connection to a viewer 23, such as elastically-loaded pins or buttons 36 or the like in one among the junction component 34 and the viewer 23 (preferably at the base thereof) and one or more openings or the like for removably receiving one or a respective elastically-loaded pin or button 36 of the other among viewer 23 or junction component 34.

According to the not limiting embodiment shown in the figures, the first end 34a of the junction component 34 delimits a pivoting seat 34e, e.g. at least spherically or hemi-spherically shaped for receiving the second end 26b or the first end 34a is at least partly spherically or hemi-spherically shaped for engaging an at least partly spherically or hemi-spherically shaped seat delimited by the second end 26b , optionally spherically or hemi-spherically shaped, of the housing component 26.

The visualization means is kept in a stable and adjustable position with respect to the housing component owing to the friction generated between the second end of the housing component and the end of the visualization means or the junction component engaged therewith.

In this respect, according to the not limiting embodiment shown in the figures, the size of the pivoting seat 34e substantially corresponds or is slightly larger than the outer encumbrance of the second end 26b of the housing component 26, in such a way as the junction component 34 is kept is a stable and adjustable position with respect to the housing component 26 owing to the friction generated between the second end 26b and the wall/s of the junction component 34 delimiting the pivoting seat 34e.

To this regards, a gasket can be provided between the second end 26b of the housing component 26 and the wall/s of the junction component 34 delimiting the pivoting seat 34e, which gasket would be designed to improve the friction among these components 26, 34. Such a gasket could be housed in a recess or annular recess 26f delimited by the housing component 26 at the second end 26b thereof.

The second end 34b of the junction component 34 is designed to be fitted, for example snap-fitted or fitted to size into a respective opening delimited by the viewer 23, for example at the base of the viewer 23.

The control board 33 can be housed and constrained in the second end 34b of the junction component 34 and the latter can delimit a spline or the like 35 designed to make it possible the electrical connection of the control board 33 with the viewer 23.

Moreover, the second end 26b of the housing component 26 can delimit a through opening 26g for the passage of the viewing means 8 and lighting means 9 extending from the control board 33.

With reference to the not limiting embodiment shown in the figures, the junction component 34 include two hemi-shells 34c, 34d to be connected with one another, for example through snap-fitting means, screws or the like.

The tube of the tubular catheter 5 can be made from plastic material suitable for medical use, for example medical PVC (polyvinylchloride) or silicone.

Thanks to the present invention, the operator can view a visualization to identify the position of the end of the catheter and the artificial airways, the trachea and the bronchial tree.

This offers advantages with respect to the state of the art. With the visualization, it is possible to ensure that the catheter is adjacent to or inside collections of liquid secretions or other material and then apply the suction.

The suction can be applied selectively in order to avoid lesions to the mucosa and to facilitate the more complete elimination of the secretions.

The catheter according to the present invention also allows viewing to diagnose intra-bronchial and tracheal pathologies and allows the accumulation of secretions in the artificial respiratory ways to be identified.

The present invention has been described according to preferred embodiments, but equivalent variants can be devised without departing from the scope of protection offered by the claims.

What is claimed is:

1. A catheter unit for tracheo-bronchial suction comprising a tubular catheter, in particular of the disposable type, and equipped with an opening able to be connected to suction means through a channel, wherein said tubular catheter is provided with at least one reinforcement mesh or grid.

2. The catheter unit according to claim 1, wherein said at least one reinforcement mesh is hidden or not visible or revealable outside said tubular catheter.

3. The catheter unit according to claim 1, wherein said at least one reinforcement mesh is fitted in said tubular catheter or inside the same, i.e. embedded therein or in the matrix thereof.

4. The catheter unit according to claim 1, wherein said at least one reinforcement mesh is shaped as a tubular member with axis substantially corresponding to the main longitudinal axis of said tubular catheter.

5. The catheter unit according to claim 1, wherein said at least one reinforcement mesh is extruded together with the matrix forming said tubular catheter.

6. The catheter unit according to claim 1, wherein said tubular catheter includes viewing means and lighting means, and wherein said catheter unit delimits two channels only, i.e. a first channel designed to be connected to suction means suitable for sucking up fluid secretions and/or other similar material that can be found in a patient, and a second channel housing said viewing means and lighting means.

7. A catheter unit for tracheo-bronchial suction comprising a tubular catheter, in particular of the disposable type, and equipped with an opening able to be connected to suction means through a channel, wherein said catheter unit includes a handle comprising a housing component delimiting a main seat for receiving a connector component, and delimiting at a first end thereof an insertion opening for the insertion of said tubular catheter, wherein said connector component includes a tubular member comprising a main tubular body having a first end of insertion and constrain of the tubular catheter and a second end to be connected in fluid communication with suction means.

8. The catheter unit according to claim 7, wherein said connector component comprises a branch tubular body extending from the main tubular body and including a first extremity opening at the through hole delimited by the main tubular body and a second extremity, which, in use, opens outside the housing component and can be closed up by an operator as soon as the suction means are activated or slightly before or after such a step.

9. The catheter unit according to claim 7, wherein said connector component is provided with a bushing component to be fitted or fixed into the first end of said main tubular body and designed to clamp a respective proximal end of said tubular catheter.

10. The catheter unit according to claim 9, wherein said bushing component is wholly or partly tapered with cross section decreasing in a direction from the first end to the second end of said main tubular body, i.e. in the direction of insertion or fitting with the main tubular body.

11. The catheter unit according to claim 9, wherein said bushing component has a C-shaped cross-section for whole or part of its extension and delimits a groove extending from one end to an intermediate part thereof.

12. The catheter unit according to claim 9, wherein said bushing component includes a proximal or base wall to be firstly inserted or fitted into the first end of the main tubular body or distal from the distal end of the tubular catheter and a side tubular wall protruding from the proximal or base wall and integral or made enbloc therewith, the free end of the side tubular wall defining an open or through end of said bushing component, and wherein the side tubular wall delimits a through longitudinal light designed to house or enclose the proximal end of the tubular catheter, whereas the proximal or base wall delimits a transfer through hole with a section corresponding to and entirely aligned, in use, only with the first channel delimited by tubular catheter designed to be connected to suction means and suitable for sucking up fluid secretions and/or other similar material that can be found in a patient, so that the connector component or better yet the through hole delimited by the main tubular body is in fluid communication with the first channel only delimited by the tubular catheter.

13. The catheter unit according to claim 12, wherein said tubular catheter includes viewing means and lighting means, and wherein said catheter unit delimits two channels only, i.e. said first channel designed to be connected to suction means suitable for sucking up fluid secretions and/or other similar material that can be found in a patient, and a second channel housing said viewing means and lighting means.

14. The catheter unit according to claim 7, wherein viewing means and lighting means are provided within the seat of the housing component and outside the connector component, wherein said catheter unit comprises at least one control board, and wherein said viewing means and said lighting means extend from said at least one control board into the tubular catheter and up to the distal end thereof.

15. The catheter unit according to claim 14, wherein said viewing means include a cable or an optical sensor, whereas said lighting means include one or more optical fibers surrounding or running around or flanked to the viewing means for the whole extension thereof.

16. The catheter unit according to claim 15, wherein the group or bundle including viewing means and lighting means is entered into the proximal end of the tubular catheter passing throughout the seat delimited by said housing component.

17. The catheter unit according to claim 16, wherein said group or bundle is entered into the proximal end of the tubular catheter through a groove of a bushing component of said connector component.

18. The catheter unit according to claim 7, wherein said catheter unit further includes visualization means able to be separated and connected to said tubular catheter through connection means.

19. A catheter unit for tracheo-bronchial suction comprising a tubular catheter, in particular of the disposable type, and equipped with an opening able to be connected to suction means through a channel, said catheter unit further including visualization means able to be separated and connected to said tubular catheter through connection means, wherein said catheter unit comprises a handle suitable for giving rotations and movements to the tubular catheter, and wherein the connection of said handle to the visualization means is obtained through a pivoting or ball or spherical joint.

20. The catheter unit according to claim 19, wherein said handle comprises a housing component delimiting a main seat for receiving a connector component, and delimiting at a first end thereof an insertion opening for the insertion of said tubular catheter, whereas its other or second end is suitable for being connected to said visualization means or to a junction component for the connection to said visualization means, said second end of said housing component being at least partly spherically or hemi-spherically shaped or delimiting an at least partly spherically or hemi-spherically shaped pivoting seat for the connection to a respective end of the visualization means or of a junction component provided between the housing component and the visualization means.

21. The catheter unit according to claim 19, wherein the visualization means is kept in a stable and adjustable position with respect to the housing component owing to the friction generated between the second end of the housing component and end of the visualization means or junction component engaged therewith.

22. The catheter unit according to claim 19, wherein said connector component includes a tubular member comprising a main tubular body having a first end of insertion and constrain of the tubular catheter and a second end to be connected in fluid communication with suction means and a branch tubular body extending from the main tubular body and including a first extremity opening at the through hole delimited by the main tubular body and a second extremity, which, in use, opens outside the housing component and can be closed up by an operator as soon as the suction means are activated or slightly before or after such a step.
